(19) United States
(12) Patent Application Publication
(10) Pub. No.: US 2004/0116832 A1
(43) Pub. Date: Jun. 17, 2004

(54) Catheter arrangement

(75) Inventors: Holger Friedrich, Dresden (DE); Raimund Erbel, Essen (DE)

Correspondence Address: GUDRUN E. HUCKETT, LONSSTR. 53, WUPPERTAL, 42289, DE

(73) Assignee: curative medical devices GmbH, Dresden (DE)

(21) Appl. No.: 10/464,843

(22) Filed: Jun. 19, 2003

(30) Foreign Application Priority Data
Jun. 21, 2002 (DE) 102 27 893.8

(51) Int. Cl. A61B 5/00; A61M 25/01

(52) U.S. Cl. 600/585

(57) ABSTRACT

The invention relates to a catheter arrangement for invasive medical treatment for introducing and controlling a guide wire in a vessel branch or vessel entry by bending the guide wire. The catheter arrangement comprises two catheters, each having a distal end and a proximal end and a lumen, wherein the two catheters are embodied as an inner catheter 3 and an outer catheter 2 extending coaxially within one another and arranged so as to be moveable relative to one another in an axial direction, wherein the inner catheter 3 has a pre-bent distal tip 3a of a bendable material and the outer catheter 2 has a continuous lumen 22 extending from the proximal end 20 to the distal end 21 for receiving the inner catheter 3, wherein both catheters 2, 3, at a spacing from the distal end 21, have lateral openings 23, 33 for passing the guide wire F therethrough.

The invention relates to a catheter arrangement for an invasive medical treatment for introducing a guide wire into a vessel branch or vessel entry by bending the guide wire, comprising two coaxially arranged catheters that are movably guided within one another. The invention is suitable in particular for placing a guide wire into a branch vessel that branches off a main vessel of the coronary system within a range of magnitude of more than 90°.

It is known and described in the pertinent technical literature and patent literature to employ usually balloon catheters and/or stents for typical treatments of coronary vessel constrictions, wherein, as a matter of routine, this is carried out such that the puncture is realized either within the groin area or in the bend of the elbow and that in this way the artery system is made accessible. Through the respective puncture location a so-called guide catheter in the form of a pre-shaped tube is introduced that guides the future treatment component into a position in the vicinity of the aorta valve upstream of the branch of the coronary vessels branching away from the aorta. After the guide catheter has been moved to the desired location while being checked by X-ray, a guide wire is subsequently inserted that is extremely flexible at the tip. The guide wire is then introduced into the coronary vessel and, if possible, moved past the constricted location; this is made possible, for example, by prebending the tip and carrying out externally a corresponding rotation as well as manipulation. However, with regularity problems are frequently encountered in regard to placement, in particular, when the target vessel in the coronary vessel system is positioned relative to the main vessel, for example, at an angle of more than 90°. In these cases, it is often not possible to introduce the guide wire with corresponding pressure into the branch vessel branching off the main vessel. European patent 237 564 discloses, for example, a guide catheter that can be mechanically positioned at an angle. It comprises an anchoring element and an actuating element as well as a flexible element that is connected at one side with a reinforcement element preventing longitudinal expansion. The flexible element is fixedly connected at its proximal end to the anchoring element and at its distal end to the actuating element. When the actuating element is moved in the direction of the proximal end, the flexible element is contracted in the direction toward the anchoring element and is angled as a result of the one-sided reinforcement. A disadvantage of the guide catheter disclosed in European patent 237 564 is its complicated construction. A further disadvantage is that this catheter requires a guide wire that is at least twice as long as the catheter itself. This length complicates sterile manipulation and also entrains logistic problems because conventionally employed guide wires are not long enough.

It is an object of the invention to provide a medical device having a simple constructive configuration that enables to better position a guide wire in vessels having an anatomically difficult position and to better introduce a guide wire into branches of vessels that are bent relative to the main vessel by 90° and more. In particular, by means of this device a placement of guide wires that are only insignificantly longer than the device itself should be possible within vessels having an anatomically difficult position.

According to the invention this object is solved with the features of the independent claim in that the catheter arrangement for the invasive medical treatment for introducing and controlling a guide wire in a vessel branch or vessel entry by bending the guide wire comprises two catheters having a distal end and a proximal end and a lumen, wherein the two catheters are configured as an inner catheter 3 and an outer catheter 2 that are arranged coaxially within one another and are movable relative to one another in the axial direction. For this purpose, the outer catheter 2 has a continuous lumen (=cavity) extending from its proximal end 20 to its distal end 21 for receiving the inner catheter 3.

The inner catheter 3 has a pre-bent distal tip 3a comprised of a bendable material.

For passing through the guide wire, both catheters 2, 3 have lateral openings 23, 33, respectively, at a spacing from the distal end 21 for passing the guide wire F through.

The inner catheter 3 is provided at the distal area with a lumen beginning at the distal end having an outlet opening for threading the guide wire, wherein the lumen in the direction toward the proximal end extends at least to passage 33 formed in the circumference of the inner catheter from where the guide wire can be passed outwardly so that a complete threading of the guide wire can be prevented.

Before its use, the catheter arrangement is threaded with the distal end onto the guide wire F so that the guide wire exits again through the lateral opening 23 in the outer catheter and the remainder of the guide wire extends parallel to the catheter. Since the catheter therefore must not be completely threaded onto the guide wire F, a sterile manipulation of the guide wire is significantly simplified in this way.

Because of the lateral opening, the guide wire must only be insignificantly longer than the catheter itself. This is an important advantage because the presently used guide wires are relatively short; for example, usually guide wires having a length of between 1.35 to 1.9 m are used in coronary angiography.

Because of the lateral opening of the catheter arrangement, it is in particular made possible that the catheter arrangement can be threaded onto a guide wire that has already been inserted into the patient and projects only a few centimeters from the body.

This is an advantage when during surgery the surgeon is confronted with difficulties when placing the guide wire, these difficulties residing in the anatomy of the patient. This is usually not foreseeable during preoperative preparation of surgery so that usually only during the course of the surgery a decision is made to place a guide catheter onto the guide wire.

The catheter arrangement according to the invention enables in this case the subsequent threading of the catheter onto the guide wire that has not yet been correctly positioned without the guide wire having to be exchanged or removed from the body. This provides for a simple later placement of the guide wire, should the need arise.

With the catheter arrangement according to the invention, the introduction and control of the guide wire in vessels having an anatomically difficult position, in particular, in acutely angled branches S bent at an angle of a magnitude of more than 90° relative to the main vessel G, is enabled by means of the pre-bent distal tip.

Advantageous embodiments of the invention can be taken from the characterizing features of the dependent claims.

Preferably, the passages 33 of the inner catheter 3 and the lateral opening 23 of the outer catheter 2 are arranged such in the catheters 2, 3 that, in the position with the distal tip 3a retracted or extended, the openings 23, 33 are at least in partial or aligned overlapped position.

By means of the thus communicating openings 23, 33 in the inner and outer catheter 2, 3, an optimal threading of the guide wire F that can exit laterally is achieved without kinks. By means of this arrangement of the openings 23, 33, it is also ensured that the inner catheter can be moved relative to the outer catheter when the catheter arrangement is threaded onto a guide wire F.

According to a proposed configuration of the invention, when retracting the inner catheter into the outer catheter the pre-bent distal tip of the inner catheter is stretched. Upon pushing the distal tip out of distal end of the outer catheter, the distal tip returns into the preset pre-bent configuration. In this way, it is possible to thread the catheter arrangement onto the guide wire end that is outside of the body and to move it by means of the guide wire to the problem location wherein the distal tip of the inner catheter reaches by corresponding rotations the desired location in order to introduce in this way the guide wire into the branch vessel S to be treated.

In an advantageous embodiment, the inner catheter 3 in the distal area is formed with a lumen 32 and an outlet opening 32a, wherein the lumen, beginning at the distal end 31, extends in the direction toward the proximal end 30 at least up to the passage 33 formed in the circumference of the inner catheter 3.

It was found to be advantageous when the bendably designed distal tip 3a of the inner catheter 3 is configured to be bent at an angle a of approximately 60° to 120° relative to the longitudinal axis X of the catheter shaft in order to better be able to guide and position the guide wire at difficult branch locations or branches S of vessels G.

The length L1 of the lateral opening 23 of the outer catheter 2 is preferably at least as long as the length A1 of the bent distal catheter tip 3a of the inner catheter 3. In this way, an overlapped positioning of the lateral openings 23, 33 of the inner and outer catheters 3, 2 in the position with retracted and extended tip 3a is made possible. Advantageously, in this way the passage of the guide wire F in both positions is enabled and friction between the guide wire and the two catheters 2,3 is minimized.

The spacing A4 of the lateral opening 23 in the outer catheter 2 from the distal end 21 is preferably greater or identical to the length L1 of the lateral opening 23.

Preferably, the lateral opening 23 of the outer catheter 2 is in the form of an elongate hole, a slotted hole-shaped lateral outlet, elliptical or oval. The passage 33 of the inner catheter 3 is preferably circular. In this connection, the lateral opening 23 of the outer catheter is greater than the passage of the inner catheter.

In another configuration, the inner catheter and the outer catheter are provided with an X-ray opaque marking near the distal end, respectively. The markings 24 make it possible that the treating physician can determine precisely where the catheter arrangement with the pre-bent catheter tip is located.

The inner catheter 3 and the outer catheter 2 are preferably configured of torsionally stiff materials with approximately identical stiffness. Advantageously, by means of the materials with approximately identical stiffness a rotation of the catheter relative to one another when introducing the catheter arrangement is prevented.

Preferably, the area of the lateral opening 23 of the outer catheter 2 is comprised of a bending-resistant or torsionally stiff material or reinforced with such material in this area. The reinforcement of the lateral opening ensures the required bending and torsional stiffness and prevents kinking in the area of the opening. The reinforcement is realized preferably by means of a metal sleeve, a metal mesh or a plastic mesh.

The material of the distal tip 3a of the inner catheter 3 is comprised advantageously of a memory material, for example, Nitinol or a heat-treated plastic material. These materials enable to bend the tip during manufacture into a pre-shaped configuration and the tip returns into this shape when it is extended from the distal end 21 of the outer catheter 3.

In another advantageous configuration, the catheter arrangement is provided at the proximal end with a manipulation instrument comprising a control mechanism. This manipulation instrument serves for retracting and extending the distal tip 3a of the inner catheter by movement of the inner catheter in the axial direction relative to the outer catheter.

Moreover, the proximal end of the outer catheter 2 preferably comprises a guide for the proximal end of the inner catheter 3. The guide enables the axial movement of the catheters relative to one another. At the same time, the guide prevents a radial rotation of the catheters relative to one another and ensures in this way that the catheters 2, 3 can be moved into a position in which the lateral openings 23, 33 are at least partially overlapped.

Such a guide is realized, for example, in that the outer and inner catheters are configured as shape-ideal counter members in the area of their proximal ends.

The invention will be explained in more detail in the following with the aid of the drawings and one embodiment. It is shown in:

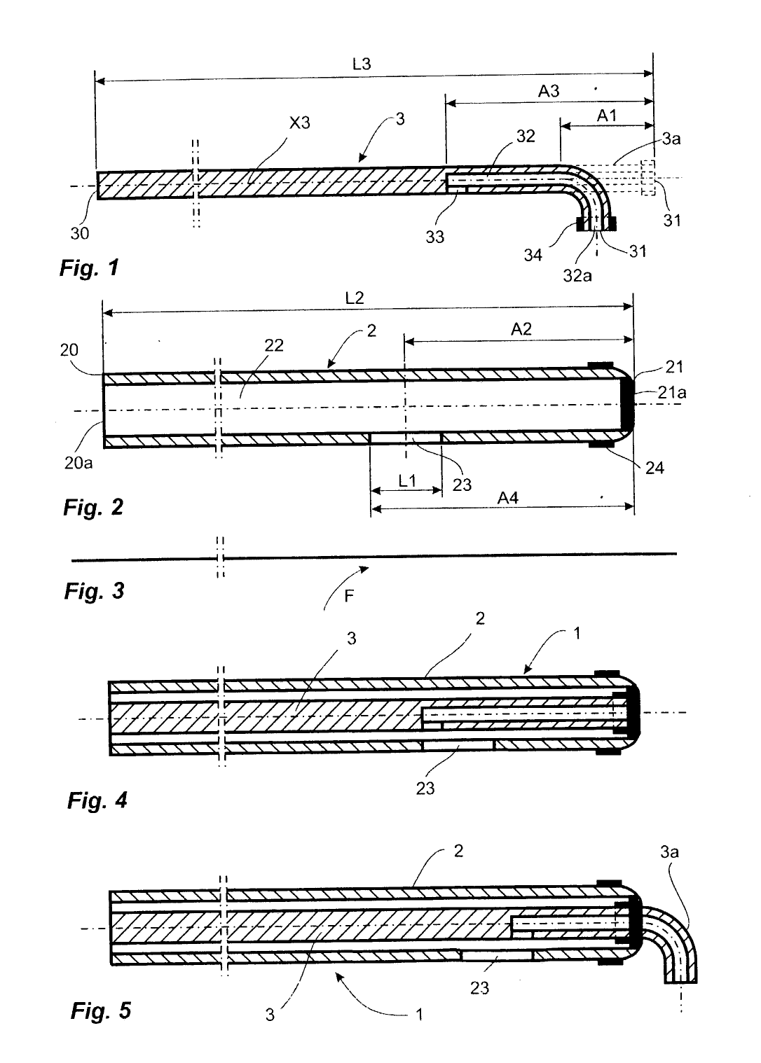

FIG. 1 a schematic longitudinal section of the inner catheter;

FIG. 2 a schematic longitudinal section of the outer catheter;

FIG. 3 a guide wire;

FIG. 4 a schematic longitudinal section of the catheter arrangement with retracted inner catheter;

FIG. 5 a schematic longitudinal section of the catheter arrangement with extended catheter tip;

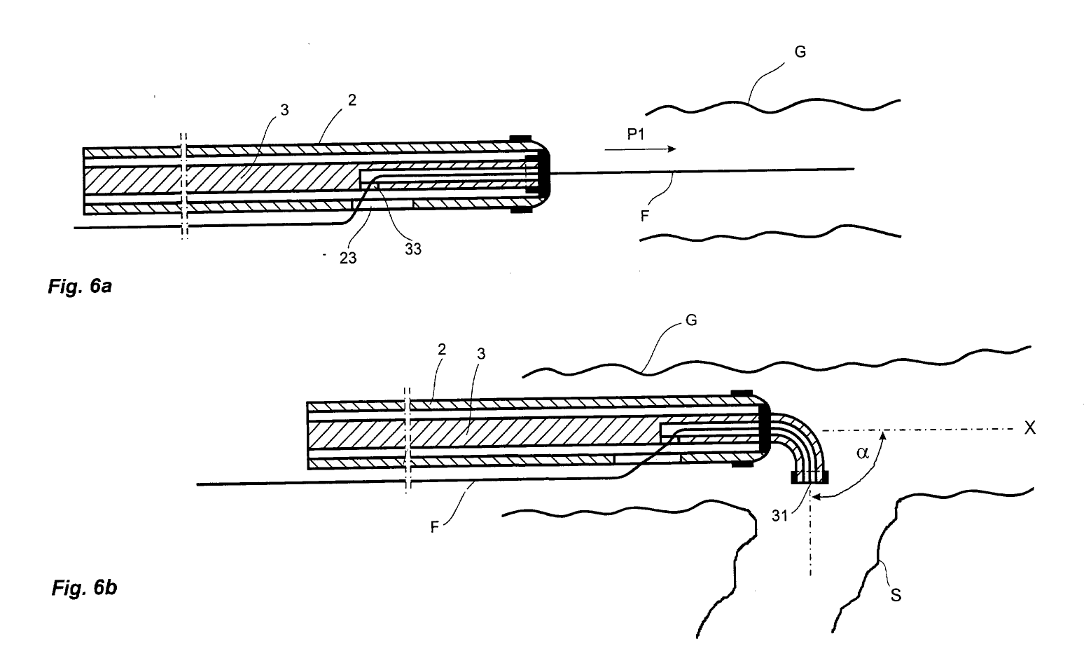

FIG. 6a and

FIG. 6b a schematic illustration of the catheter arrangement for threading and inserting a guide wire into a vessel;

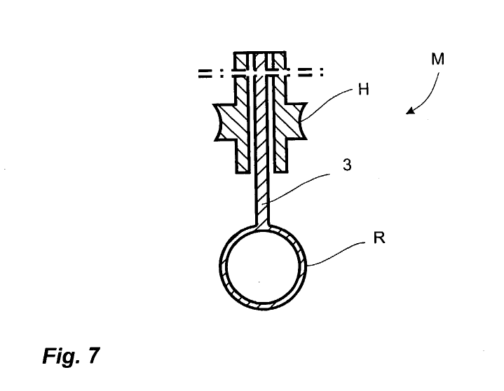

FIG. 7 a schematic illustration of a manipulation instrument.

The catheter arrangement 1 according to the invention illustrated in FIGS. 1 through comprises two catheters 2, 3, i.e., an outer thicker catheter 2 (see FIG. 2) and an inner thinner catheter 3 (see FIG. 1). The inner catheter 3 is longer in comparison to the outer catheter 2, i.e., it projects past the outer catheter 3. The materials to be used for both catheters are of almost identical stiffness. It is moreover important that the catheter materials have in particular a certain torsional stability. The inner catheter 3 has at its distal end—the catheter tip—a bent end section, i.e., the distal tip 3a is bendably pre-shaped and provided with a memory material or changeable material. For example, as a memory material Nitinol or heat-treated plastic material, that in a certain way has memory properties and maintains at least for the duration that is required here these memory properties, can be used. The pre-bent distal tip of the inner catheter 3 is approximately 7 mm long, corresponds to the length A1 and has an outlet opening 32a for the guide wire F to be received. Both catheters 2, 3 are delivered in their inserted state, i.e., they are arranged coaxially within one another and movable in a telescoping way, wherein the distal tip 3a of the inner catheter 3 in the delivered state is pre-bent and projects at an angle α of approximately 90° past the catheter periphery of the outer catheter 2. For threading the guide wire F, the inner catheter 3 is retracted by the tip length A1. In this connection, the pre-bent distal tip 3a is stretched and secured in the outer catheter 2 in a straight shape (see FIGS. 4 and 6a). The catheter arrangement is adjustable as its own body, i.e., both catheters 2, 3 can be moved relative to one another. Moreover, the lumen 22 of the outer catheter 2 is configured to be continuous from the proximal end 20 to the distal end 21 and is provided with an inlet opening 20a and with an outlet opening 21 a (see FIG. 2). The lumen 32 of the inner catheter 3, on the other hand, is not continuous, as shown in FIG. 1, and extends only from the outlet opening 32a at the distal end 31 to a point inclusive the passage 33, i.e., opens into this opening at the circumference of the catheter shaft. The passage 33 is positioned at a spacing A3 from the stretched distal end 31 of the inner catheter 3 and serves for providing a fast threading action of the guide wire F that projects laterally outwardly and therefore must not be completely threaded—this is referred to as monorail or fast exchange technique and, in turn, requires that both catheters 2, 3 have an opening, respectively, that at least partially overlap and communicate with one another (see FIGS. 6a, 6b). The passage 33 of the inner catheter is circular. The outer catheter 2 has at the catheter circumference a slotted hole-shaped lateral outlet 23 that is connected to the lumen 22. The lateral outlet 23 has the same length as the movable portion of the inner catheter 3 and the pre-bent tip of the inner catheter 3, i.e., the spacing A4 of the slotted hole-shaped lateral outlet 23 relative to the distal end 21 of the outer catheter is as long as A3 plus L1; however, at least half of A3. In this way, it is ensured that the guide wire F can exit laterally and, in spite of this, the inner catheter 3 can be moved relative to the outer catheter 2. In order to enable passing of the guide wire F and relative movement of the coaxially positioned catheters 2, 3, the passage 33 of the inner catheter 3 must match with regard to its diameter the lumen 32 of the inner catheter and the slotted hole-shaped, oval lateral outlet 23 must be provided in the outer catheter 2. The passage 33 must therefore be positioned in the area of the slotted hole-shaped lateral outlet 23 wherein the passage 33 and the slotted hole-shaped lateral outlet 23 are arranged such that for a tip-level arrangement of both catheters 2, 3 or for an extended pre-bent distal tip 3a overlap at least in partial areas. Since for threading of the guide wire F the inner catheter must be retracted into the outer catheter 2 (see FIGS. 4a, 6a) and subsequently must be moved out, the length L1 of the slotted hole-shaped lateral outlet 23 must be at least as long as the length A1 of the bent distal catheter tip 3a. At the distal end 20, 30 of the catheter shaft—of the outer catheter 2 as well as the inner catheter 3—a manipulation instrument (M) with control mechanism is provided, wherein a part of the manipulation mechanism, i.e., a handle (H), is provided fixedly on the outer catheter 2 and another part on the inner catheter 3. The manipulation instrument (M) serves for moving the outer catheter (2) relative to the inner catheter (3). For example, the outer catheter 2 is held by the index and middle fingers and the inner catheter 3 is moved back and forth by the thumb, wherein especially for the thumb an annular part (R) is provided (see FIG. 7). Both catheters 2, 3 are provided in the area of the distal ends 21, 31 with a marking 24, 34 that is X-ray opaque.

In invasive medical treatments it is, for example, necessary to introduce a guide wire into a branch having a bend of approximately 90°. First, the guide wire that optionally has a pre-bent tip, is introduced, for example, by means of a standard guide catheter having a continuous lumen as far as possible into the coronary vessel system, if possible, up to the location of the constricted area, and is left there. The treating physician determines that a difficult bend/curve cannot be circumnavigated and the guide wire cannot be introduced as usual into the target vessel. In this case, the coaxial guide catheter is employed. The coaxial guide catheter 1 is threaded at the catheter tip via the outlet opening 32a of the inner catheter 3 onto the guide wire end located outside of the body in the direction of arrow P according to FIG. 6a. The guide wire end is then moved outwardly via the passage 33 of the inner catheter 3 and the slotted hole-shaped lateral outlet 23 and the guide catheter is moved across the provided guide wire to such an extent that the two coaxially guided catheters 2 and 3 can be moved to the difficult location to be treated, for example, a location within the vessel branch. The inner catheter is then moved relative to the outer one wherein the pre-bent distal tip 3a of the inner catheter 3 points approximately at 90° away from the longitudinal axis and is brought by corresponding rotations to the location in question (see FIG. 6b). By means of the pre-bent tip 3a, the guide wire can be introduced into the corresponding vessel or branch vessel. The guide wire extends thus within the inner catheter 3, exits to the exterior, and extends parallel to both catheters 2, 3 (see FIG. 6b).

### List of Reference Numerals

F=guide wire

1=catheter arrangement

2=outer catheter

20=proximal end of outer catheter 2

20a inlet opening at the distal end 20

21=distal end of the outer catheter 2

21a=outlet opening at the distal end 21

22=continuous lumen of the outer catheter 2

23=lateral opening at the periphery of the outer catheter 2

24=X-ray detectable marking at the distal end 21

3=inner catheter

3a=bendable distal tip of the catheter 3

30=proximal end of the inner catheter 3

31=distal end of the inner catheter 3

32=lumen of the inner catheter 3

32a=exit opening of the lumen 32 at the distal end 31

33=lateral passage at the periphery of the inner catheter 3

34=X-ray detectable marking at the distal end 31

X=longitudinal axis of the catheter 3

α=angle between longitudinal axis X and distal end 31 of the inner catheter 3

A1=length of the bent distal catheter tip 3a

A2=spacing of the center of the lateral opening 23 of the outer catheter 2 relative to the distal end 21 of the outer catheter 2

A3=spacing of the passage 33 relative to the stretched distal end 31 of the inner catheter 3

A4=spacing of the lateral opening 23 of the outer catheter 2 relative to the distal end 21

L1=length of the lateral opening 23 of the outer catheter 2

L3=total length of the inner catheter

L2=length of the outer catheter

G=coronary vessel

M=manipulation instrument

R=annular part

S=branch vessel

1. A catheter arrangement for invasive medical treatment for introducing and controlling a guide wire (F) in a vessel branch or vessel entry by bending the guide wire, comprised of two catheters, each having a distal end and a proximal end and a lumen, wherein the two catheters are embodied as an inner catheter (3) and an outer catheter (2) extending coaxially within one another and arranged so as to be moveable relative to one another in an axial direction, wherein the inner catheter (3) has a pre-bent distal tip (3a) of a bendable material and the outer catheter (2) has a continuous lumen (22) extending from the proximal end (20) to the distal end (21) for receiving the inner catheter (3), wherein both catheters (2, 3), at a spacing from the distal end (21), have lateral openings (23, 33) for passing the guide wire (F) therethrough.

2. The catheter arrangement according to claim 1, characterized in that the passage (33) of the inner catheter (3) and the lateral opening (23) of the outer catheter (2) are arranged such in the catheters (2, 3) that in the position with retracted or extended distal tip (3a) the openings (23, 33) are at least partially overlapping one another.

3. The catheter arrangement according to claim 1 or 2, characterized in that, when retracting the inner catheter (3) into the outer catheter (2), the pre-bent distal tip (3a) of the inner catheter (3) can be stretched and, when extending the distal tip (3a) from the distal end (21) of the outer catheter (2), the distal tip returns into the preset pre-bent shape.

4. The catheter arrangement according to one of the claims 1 through 3, characterized in that the inner catheter (3) in the distal area is provided with a lumen (32) beginning at the distal end (31) having an exit opening (32a), wherein the lumen (32) extends in the direction toward the proximal end (30) up to at least a passage (33) formed in the circumference of the inner catheter (3).

5. The catheter arrangement according to one of the claims 1 through 4, characterized in that the distal tip (3a) of the inner catheter (2) is pre-bent at an angle (α) of approximately 60° to 120° relative to the longitudinal axis (X) of the catheter shaft.

6. The catheter arrangement according to one of the claims 1 through 5, characterized in that the length (L1) of the lateral opening (23) of the outer catheter (2) is at least as long as the length (A1) of the bent distal catheter tip (3a) of the inner catheter (3).

7. The catheter arrangement according to one of the claims 1 through 6, characterized in that the spacing (A4) of the lateral opening (23) in the outer catheter (2) from the distal ends (21) is greater or the same as the length (L1) of the lateral opening (23).

8. The catheter arrangement according to one of the claims 1 through 7, characterized in that the lateral opening (23) of the outer catheter (2) is configured in the form of a slotted hole or oval and the passage (33) of the inner catheter (3) is circular.

9. The catheter arrangement according to one of the claims 1 through 8, characterized in that the inner catheter (3) and the outer catheter (2) near the distal end are marked with a material that is detectable by X-rays.

10. The catheter arrangement according to one of the claims 1 through 9, characterized in that the inner catheter (3) and the outer catheter (2) are comprised of torsionally stable material with approximately identical stiffness.

11. The catheter arrangement according to one of the claims 1 through 10, characterized in that the outer catheter (2) in the area of the lateral opening (23) is made of a bending-resistant and/or torsionally stiff material or is reinforced with such a material.

12. The catheter arrangement according to claim 11, characterized in that the outer catheter (2) is reinforced in the area of the lateral opening (23) with a sleeve or a mesh of metal and/or plastic material.

13. The catheter arrangement according to one of the claims 1 through 12, characterized in that the distal tip (3a) of the inner catheter is comprised of a memory material, preferably Nitinol, or a heat-treated plastic material.

14. The catheter arrangement according to one of the claims 1 through 13, characterized in that, at a proximal end, it has a manipulation instrument (M) comprising a control mechanism for moving the inner catheter (2) within the outer catheter (3) in the axial direction, wherein the proximal end of the outer catheter (2) provides a guide for the proximal end of the inner catheter (3) that prevents a radial rotation of the catheters relative to one another.
